(19) United States
(12) Patent Application Publication
(10) Pub. No.: US 2021/0179690 A1
(43) Pub. Date: Jun. 17, 2021

(54) CTLA4-IG FUSION PROTEIN FORMULATION

(71) Applicant: DR. REDDY?S LABORATORIES LIMITED, Hyderabad (IN)

(72) Inventors: Murali JAYARAMAN, Kancheepuram (IN); Lakshmi KANAKADURGA M, Hyderabad (IN)

(73) Assignee: DR. REDDY?S LABORATORIES LIMITED, Hyderabad (IN)

(21) Appl. No.: 17/058,249

(22) PCT Filed: May 24, 2019
(86) PCT No.: PCT/IN2019/050409

§ 371 Date: Nov. 24, 2020

(30) Foreign Application Priority Data
May 25, 2018 (IN) 201841019606

(51) Int. Cl. C07K 14/705; A61K 47/18; A61K 47/26

(52) U.S. Cl. CPC C07K 14/70521; A61K 47/183; A61K 38/00; C07K 2319/30; C07K 2319/32; A61K 47/26

(57) ABSTRACT

The present invention discloses a stable pharmaceutical formulation of a fusion protein, wherein the formulation contains buffer, sugar alcohol/polyol, amino acid and surfactant, and wherein the formulation is devoid of sucrose. Additionally, the formulation may also be devoid of a salt. The disclosed fusion protein formulations are liquid formulations that are also suitable for lyophilization.

### FIELD OF INVENTION

The present invention is related to stable formulation of a fusion protein molecule. In particular, the invention discloses stable cytotoxic T-lymphocyte-associated protein 4-immunoglobulin (CTLA4-Ig) fusion protein formulation, wherein the formulation comprises buffer systems and stabilizers.

### BACKGROUND

Over the past two decades, recombinant DNA technology has led to the commercialization of many proteins, particularly antibody therapeutics and fusion protein molecules.

Fusion proteins, in particular, Fc fusion protein molecules (in which Fc portion of human immunoglobulin (Ig) is conjugated to a particular portion of a receptor) are gaining significance, since their wide usage in treatment of various oncological and immunological disorders. Etanercept (TNFR-IgFc), aflibercept (VEGFR-IgFc) and abatacept and belatacept (CTLA4-IgFc) are among those Fc fusion proteins approved by Food and Drug Administration (FDA) to treat various disorders. The effectiveness of fusion protein molecule is majorly dependent on the stability, route of administration and their dosage forms and concentrations. This in turn, necessitates these protein molecules to be formulated appropriately to retain stability and activity.

Proteins in general, and Fc fusion proteins in particular, are typically unstable in solution and sensitive to pH, temperature and oxidation and hence can undergo a variety of covalent and non-covalent reactions, modifications or degradations in solution. The more common protein degradation pathways include aggregation, deamidation and/or oxidation and these degradation pathways are known to be influenced by pH, temperature and storage conditions, including formulation conditions and excipients. These pathways thus lead to both physical and chemical instability of a protein in solution.

Aggregation in therapeutic proteins, is of particular interest, because it often results in decreased bioactivity/loss of activity over a period of time and may be immunogenic when administered to a patient. In case of fusion proteins aggregation is significant since they involve fusion of two or more proteins, are large and complex structure and tend to form aggregates at a rapid rate as compared to simple polypeptides or antibodies.

Apart from aggregation, another type of instability of a multimeric protein, specifically occurring at the regions where two or more proteins are fused, is fragmentation/clipping which can be a result of deamidation, oxidation, isomerization and/or hydrolysis. Deamidation can occur at aspargine or glutamine residues, resulting in a charge variant/s of the protein. Oxidation of fusion proteins involves mainly methionine residues, and are generally influenced by external factors such as exposure to light and transition metal ions or degradation product of an excipient (e.g., hydrogen peroxide from polysorbate degradation). Presence of these oxidized products and charge variants in a therapeutic protein molecule are known to increase instability and, thus decrease the bioactivity of the protein.

Hence, it is essential to develop a suitable mixture of formulation component/s that would stabilize a therapeutic (fusion) protein molecule against the many physical and chemical instability inducing factors. Further, the developed formulation should maintain colloidal stability during storage conditions, since it measures and ensures that the protein molecules remain suspended in an aqueous solution at equilibrium.

There are numerous class of excipients such as sugar and sugar alcohols, amino acids and surfactants which are used in stabilizing proteins and fusion protein molecules. However, the choice of excipients while formulating a protein is governed by various other factors such as their compatibility with the protein and other components in the formulation, (intended) mode of administration and dosage of the therapeutic protein, etc. Therefore, the challenge behind a formulation development involves screening and selection of suitable buffer conditions and excipients, including their concentrations, to achieve a stable formulation. Further, it is also expected that the developed formulation is stable at room temperature and be suitable to be administered in either lyophilized or liquid form.

### SUMMARY

The present invention discloses a stable pharmaceutical formulation of a fusion protein molecule comprising buffer, polyol, amino acid and surfactant, wherein the fusion protein is a CTLA4-Ig molecule.

In particular, the invention discloses a stable pharmaceutical formulation of CTLA4-Ig fusion protein comprising buffer, polyol, amino acid and surfactant wherein the said formulation is devoid of sucrose.

The above disclosed stable formulations of CTLA4-Ig fusion protein, may optionally be free of sodium chloride.

In addition, the invention discloses a method of reducing aggregation and/or fragmentation of CTLA4-Ig fusion protein in a formulation comprising addition of histidine and polyol.

The invention also discloses a method of increasing the stability of CTLA4-Ig fusion protein formulation, comprising histidine and mannitol, wherein the histidine and mannitol components are also added during the process step, in particular in the tangential flow filtration process step (a step before the formulation step). Such addition during the process imparts significant stability to the formulation.

The disclosed CTLA4-Ig fusion protein in the said formulation is stable at lower, as well as higher concentration (from 10 mg/ml to 200 mg/ml) and at various temperatures, including at 30° C. for at least two weeks. The said formulation contains less than about 10% of the fusion protein molecules in aggregate form and preferably less than 7.5% of the fusion protein molecules in aggregate form.

Additionally, the combination of polyol and amino acid imparts colloidal stability to the fusion protein molecule present in the formulation.

### DETAILED DESCRIPTION OF THE INVENTION

### Definitions

The term “fusion protein” means a protein formed by fusing (i.e., joining) all or part of two polypeptides which are not the same. Typically, fusion proteins are made using recombinant DNA techniques, by end to end joining of polynucleotides encoding the two polypeptides.

The terms “CTLA4-Ig” or “CTLA4-Ig molecule” or “CTLA4Ig molecule” are used interchangeably, and refer to a protein molecule that comprises a polypeptide having a CTLA4 extracellular domain or a portion thereof, and an immunoglobulin constant region or a portion thereof. The extracellular domain and the immunoglobulin constant region can be wild-type, or mutant or modified, and mammalian, including human or mouse. The polypeptide can further comprise additional protein domains. A CTLA4-Ig molecule can also refer to multimer forms of the polypeptide, such as dimers, tetramers, and hexamers. A CTLA4-Ig molecule also is capable of binding to CD80 and/or CD86.

The term “stable” formulation refers to the formulation wherein the antibody therein retains its physical stability and/or chemical stability and/or biological activity, upon storage.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the fusion protein in the pharmaceutical formulations. A fusion protein “retains its physical stability” in a pharmaceutical formulation if it shows substantially no signs of aggregation, precipitation and/or denaturation upon visual examination of color and/or clarity, or as measured by UV light scattering or by size exclusion chromatography. A fusion protein is said to “retain its chemical stability” in a pharmaceutical formulation when its shows no or minimal formation of product variants which may include variants as a result of chemical modification of fusion protein such as deamination, oxidation etc. Analytical methods such as ion exchange chromatography and hydrophobic ion chromatography may be used to investigate the chemical product variants.

The monomer, dimer and high molecular weight (HMW) species of CTLA4Ig molecule may be separated by size exclusion chromatography (SEC). SEC separates molecules based on the molecular size. Separation is achieved by the differential molecular exclusion or inclusion as the molecules migrate along the length of the column. Thus, resolution increases as a function of column length. In order to maintain the appropriate activity of a fusion protein, it is desirable to reduce the formation of aggregate or fragmentation (monomer/low molecular weight species) of products and hence control the dimer content to a target value. Dimer is major form present in fusion proteins and elutes as main peak in size exclusion chromatography. CTLA4Ig molecule samples may be separated using a 2695 Alliance HPLC (Waters, Milford, Mass.) equipped with TSK Gel® G3000SWXL (300 mm×7.8 mm) and TSK Gel® G3000SWXL (40 mm×6.0 mm) columns (Tosoh Bioscience, Montgomery, Pa.).

The colloidal stability of a protein gives information on interaction of proteins molecules within self, and between the surrounding molecules, in an aqueous environment. A common indicator or predictor of colloidal stability of a protein molecule in a solution is the diffusion co-efficient (k_{D}) value, measured by dynamic light scattering (DLS) technique. The higher the diffusion co-efficient value, the more the repulsive forces, more solubility and less aggregate formation in the protein molecule, and thus the protein exhibits colloidal stability. And colloidal stability is an indicator of protein solubility, viscosity, type of protein aggregates etc.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, sugars, salts, surfactants, and derivatives and combination thereof.

Pre-formulation steps refer to any or multiple steps performed before formulating the protein into a therapeutic product. Examples of such steps include, chromatography, filtration, (ultrafiltration, sterile filtration, nano filtration, diafiltration, depth filtration), or any other steps performed to concentrate the protein or to exchange the buffer to a different/suitable buffer. The filtration steps mentioned herein may be performed in a tangential flow filtration mode.

The term “polyol” or “sugar alcohol” as used herein includes an organic compound containing multiple hydroxyl groups. Examples of polyols include mannitol, sorbitol, xylitol etc.,

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

Examples of salts include, but not limited to, sodium chloride, potassium chloride, magnesium chloride, sodium thiocyanate, ammonium thiocyanate, ammonium sulfate, ammonium chloride, calcium chloride, zinc chloride and/or sodium acetate.

Certain specific aspects and embodiments of the invention are more fully described by reference to the following examples. However, these examples should not be construed as limiting the scope of the invention in any manner.

### DETAILED DESCRIPTION OF THE EMBODIMENTS

Many of the approved fusion protein formulations contain sucrose as a stabilizer. However, the instant invention disclose a fusion protein (CTLA4-Ig) formulation which provides stability without the presence of sucrose.

The present invention discloses a stable pharmaceutical formulation of a fusion protein comprising buffer, polyol, amino acid and surfactant.

In one embodiment, the invention discloses a stable pharmaceutical formulation of a CTLA4-Ig fusion protein comprising buffer, polyol, amino acid and surfactant, and wherein the formulation is devoid of sucrose.

In one embodiment, the invention discloses a stable pharmaceutical formulation of a CTLA4-Ig fusion protein comprising buffer, mannitol, histidine and surfactant, and wherein the formulation is devoid of sucrose.

In any of above mentioned embodiments, the ratio of CTLA4-Ig fusion protein to polyol is 1:0.7 or less and the ratio of CTLA4-Ig fusion protein to amino acid is 1:0.1 or less.

In the above said embodiments of the invention, the formulation of CTLA4-Ig fusion protein also does not require salt to stabilize the therapeutic protein formulation.

In the above mentioned embodiments, the CTLA4-Ig fusion protein formulation additionally be free of salt, wherein the salt is sodium chloride.

In one embodiment, the invention discloses a stable pharmaceutical formulation of a CTLA4-Ig fusion protein consisting essentially of buffer, mannitol, histidine and surfactant.

In the above said embodiments, the concentration of polyol present in the formulation is less than about 125 mg/ml, preferably less than about 80 mg/ml, and the concentration of amino acid present in the formulation is less than about 20 mg/ml, preferably less than 15 mg/ml and more preferably 10 mg/ml.

In any of the above said embodiments, the concentration of fusion protein in the formulation is about 10 mg/ml to 200 mg/ml, preferably 20 mg/ml to 150 mg/ml, more preferably 25 mg/ml to 125 mg/ml.

In any of the above said embodiments, viscosity of the CTLA4-Ig fusion protein formulation is less than 20 cp, preferably less than 10 cp, more preferably less than 5 cp.

In any of the above mentioned embodiments of the invention, the pH of CTLA4-Ig fusion protein formulation is from 6.0-8.0, preferably 6.5 to 7.5.

In the above said embodiments, the buffer mentioned in the formulation is organic buffer, inorganic buffer and/or combinations thereof.

In any of the above mentioned embodiment of the invention, the said organic buffer is citrate buffer, succinate buffer or acetate buffer.

In yet another embodiment of the invention, the inorganic buffer mentioned in the formulation is phosphate buffer.

In any of the above mentioned embodiments, the amino acids include basic amino acid, hydrophobic amino acids and combinations thereof.

In an embodiment, the invention discloses a stable pharmaceutical formulation of CTLA4-Ig fusion protein comprising phosphate buffer, mannitol, histidine and surfactant.

In an embodiment, the invention discloses a stable pharmaceutical formulation comprising CTLA4-Ig fusion protein molecule, citrate-phosphate buffer, mannitol, histidine and poloxamer, and wherein the formulation is stable and maintains at least 94% of fusion protein molecule as a major peak, when analyzed by size exclusion chromatography.

In yet another embodiment, the invention discloses a method of reducing aggregation in CTLA4-Ig fusion protein formulation, comprising addition of histidine and mannitol to the formulation, wherein the aggregate content in the formulation is less than 7.5% of the protein, upon storage at 30° C. for 2 weeks.

In an embodiment, the invention discloses a method of inhibiting fragmentation in CTLA4-Ig fusion protein formulation, comprising addition of histidine and mannitol to the formulation, wherein the fragmented molecule content in the formulation is less than 3%, preferably less than 1% and more preferably less than 0.5% of the protein, upon storage at 30° C. for 2 weeks.

In an embodiment, the invention discloses a stable pharmaceutical formulation of CTLA4-Ig fusion protein molecule comprising, 125 mg/ml of CTLA4-Ig fusion protein, phosphate buffer, 75 mg/ml of mannitol, 15 mg/ml of histidine and 8 mg/ml of poloxamer.

In an embodiment, the invention discloses a stable pharmaceutical formulation of CTLA4-Ig fusion protein molecule comprising, 125 mg/ml of CTLA4-Ig fusion protein, phosphate buffer, 85 mg/ml of mannitol, 10 mg/ml of histidine and 8 mg/ml of poloxamer.

In another embodiment, the invention discloses a method to maintain colloidal stability of CTLA4-Ig fusion protein in a formulation, comprising addition of histidine and polyol to the formulation.

In another embodiment, the invention discloses a method of reducing oxidation of CTLA4-Ig fusion protein in a formulation, comprising addition of histidine and polyol.

In any of the above embodiments of the invention, the formulation is a stable liquid/aqueous formulation and is suitable for, and can be lyophilized as lyophilized powders. Further, the lyophilized formulation of CTLA4-Ig fusion protein can be reconstituted with appropriate diluent to achieve the liquid formulation suitable for administration.

In any of the above mentioned embodiments, the CTLA4-Ig fusion protein is abatacept or belatacept.

In any of the above said embodiments, the amino acid present in the formulation functions as a stabilizer and does not form part of a buffering agent.

In another embodiment, the invention discloses a method of increasing the stability of CTLA4-Ig fusion protein formulation comprising, buffer, polyol, histidine and surfactant, and wherein histidine and polyol are also added in the pre-formulation process step that is in the tangential flow filtration step [performed as ultrafiltration (UF) and diafiltration (DF) for product concentration and buffer exchange].

In another embodiment, the invention discloses a method of increasing the stability of the CTLA4-Ig fusion protein composition, wherein the method comprises addition of histidine and mannitol during the process step, in particular in the tangential flow filtration step. The stability of the protein molecule is found to be significantly increased in thermal and colloidal stability, when histidine and mannitol components are added in the tangential flow filtration step, specifically in the ultrafiltration and/or diafiltration step.

In yet another embodiment, the invention discloses a method of increasing the stability of CTLA4-Ig fusion protein comprising steps of expression and purification of CTLA4-Ig fusion protein; followed by concentration and/or buffer exchange of the protein by UF-DF-UF, wherein the buffer used in any of the UF-DF-UF step includes histidine and mannitol; and followed by formulation of the protein in a buffer comprising histidine, mannitol and surfactant; wherein the stability of the protein is increased compared to the formulation of the protein that was processed by UF-DF-UF steps without the inclusion of histidine and mannitol in any of its buffer.

In an embodiment, the invention discloses a method of preparing a stable high concentration CTLA4-Ig fusion protein formulation comprising;
a) obtaining purified CTLA4-Ig fusion protein molecule from chromatographic step
b) subjecting CTLA4-Ig fusion protein obtained from step a) to ultrafiltration [UF] to concentrate the protein
c) subjecting the concentrated CTLA4-Ig fusion protein obtained from step b) to diafiltration using a buffer comprising mannitol and histidine and,
d) subjecting the CTLA4-Ig fusion protein molecule from step c) to second ultrafiltration to obtain further and highly concentrated CTLA4-Ig fusion protein drug substance wherein the concentration of formulation obtained in step d) is up to 200 mg/ml and is found to be stable as measured by the standard stability studies.

The stability of the protein molecule is found to be significantly increased in thermal and colloidal stability, when histidine and mannitol components are added in the tangential flow filtration step. And addition of histidine and mannitol in the UF or DF step, results in a stable product with % HMW being consistently less than 10, even after being subjected to accelerated stability studies.

In the above mentioned embodiment, the drug substance obtained from the above process is stable and drug product prepared from the drug substance is stable under accelerated stability conditions, wherein the concentration of the drug product is up to 140 mg/ml, preferably 130 mg/ml. The addition of histidine and sugar during DF step of TFF process helps in achieving stable and soluble higher concentrations of CTLA4-Ig fusion protein molecule (upto ˜200 mg/ml) which in turn helps in preparation of desired concentration of drug product at commercial scale by simple dilution technique. This additionally saves time and resource.

In the above said embodiments, where histidine and polyol are also added to the pre-formulation process step of UF or DF, the formulated protein contains less than 10% of the protein in aggregate form, even when stored at 30° C. for at least two weeks.

The disclosed fusion protein formulations containing only amino acid/s (and no sugar or polyol), does not stabilize the protein. Whereas, addition of mannitol to the formulation containing amino acid, exhibits stabilizing effect. Further, mannitol permits the use of combination of amino acids in the formulation mixture. However, surprisingly, and to the contrary, sucrose based fusion protein (CTLA4-Ig) formulation does not exert a superior stabilization than mannitol and does not favor the use of combination of amino acids and instead exhibits a destabilizing effect on the protein.

The above disclosed formulation of CTLA4-Ig fusion protein is a stable liquid (aqueous) formulation, which can be used for parenteral administration. Parenteral administration includes intravenous, subcutaneous, intra peritoneal, intramuscular-administration or any other route of delivery generally considered to be falling under the scope of parenteral administration and as is well known to a skilled person.

The disclosed formulations of the invention uses minimal excipients and in lesser amounts to stabilize the therapeutic fusion protein molecule.

The disclosed formulations of the invention uses lesser amounts of sugar alcohol to stabilize the therapeutic fusion protein molecule. And the disclosed formulations of CTLA4-Ig fusion protein formulations comprising buffer, polyol, amino acid and surfactant are stable and can withstand multiple freeze thaw cycles and also agitation induced stress.

The disclosed invention i.e., the formulation of the fusion protein, CTLA4-Ig (abatacept) is stabilized by histidine and mannitol combination majorly. Surprisingly, addition of sucrose or any other amino acid to the formulation, indeed does not improve the stability of the fusion protein. Abatacept being a fusion protein and dimeric in nature is a complex molecule, prone to aggregation and oxidation, is however unexpectedly stabilized only by the amino acid histidine (and polyol).

### EXAMPLES

CTLA4-Ig fusion protein molecule, abatacept, suitable for storage in the present pharmaceutical composition is produced by standard methods known in the art. For example, abatacept is prepared by recombinant expression of CTLA4 fused with CH2 and CH3 portion of human IgG in a mammalian host cell such as Chinese Hamster Ovary cells. Further, the expressed abatacept is harvested and the crude harvest is subjected to standard downstream process steps that include purification, filtration and optionally dilution or concentration steps. For example, the crude harvest of abatacept may be purified using standard chromatography techniques such as affinity chromatography, ion-exchange chromatography and combinations thereof. The purified abatacept solution can additionally be subjected to one or more filtration steps, and the solution obtained is subjected to further formulation studies.

### Example 1: Screening and Selection of Suitable Buffer to Formulate Abatacept

To select suitable buffer/s for stabilizing abatacept, various buffers were prepared. 40 mg/ml of abatacept in phosphate buffer back ground obtained from downstream chromatographic process was buffer exchanged and diluted to 25 mg/ml in the respective different buffer back ground/s. Details of the formulations are given in Table 1.

All abatacept formulations were subjected for accelerated stability studies at 25° C. and 40° C. for four weeks. Post which, the samples were analyzed for high molecular weight (HMW) species and low molecular weight (LMW) species [results are shown Table 2 and 3] using size exclusion chromatography (SEC) and also checked for change in pH [Table 4] and visual inspection [Table 5].

TABLE 1
Compositions of various abatacept formulations in different buffers
as per example 1

| Sample | |
|---|---|
| Name | Composition |
| Aba-IV-1 | Abatacept 25 mg/ml, 10 mM phosphate buffer, pH 7.2 |
| Aba-IV-2 | Abatacept 25 mg/ml, 20 mM histidine buffer, pH 7.1 |
| Aba-IV-3 | Abatacept 25 mg/ml, 20 mM citrate buffer, pH 7.1 |
| Aba-IV-4 | Abatacept 25 mg/ml, 20 mM histidine-phosphate buffer, |
| | pH 7.1 |

TABLE 2
SEC data of abatacept (25 mg/ml) formulations prepared as per example 1

| | High Molecular Weight (HMW) Species | | | | | | | | | |
|---|---|---|---|---|---|---|---|---|---|---|
| | T0 | 40° C. | | | | | 25° C. | | | |
| Sample | 0 | 1 | 2 | 4 | Δ 2 | Δ 4 | 2 | 4 | Δ 2 | Δ 4 |
| Name | W | W | W | W | W | W | W | W | W | W |
| Aba-IV-1 | 26.7 | 34.9 | 37.4 | 40.4 | 10.7 | 13.7 | 29.1 | 29.7 | 2.4 | 3.0 |
| Aba-IV-2 | 26.2 | 75.9 | 77.7 | 55.8 | 51.5 | 29.6 | 28.2 | 28.6 | 2.1 | 2.4 |
| Aba-IV-3 | 27.3 | 41.7 | 41.2 | 40.4 | 13.9 | 13.1 | 32.5 | 32.5 | 5.2 | 5.2 |
| Aba-IV-4 | 27.0 | 64.1 | 50.9 | 42.8 | 23.9 | 15.8 | 30.2 | 30.9 | 3.2 | 3.9 |

W—indicates weeks,
T0 indicates ‘zero’ time point,
Δ—Delta indicates change in a value from zero time point to a specific time point

TABLE 3
SEC data of abatacept (25 mg/ml) formulations prepared
as per example 1 at 40° C. and 25° C.

| | Low Molecular Weight (LMW) Species | | | | | | | | | |
|---|---|---|---|---|---|---|---|---|---|---|
| | T0 | 40° C. | | | | | 25° C. | | | |
| Sample | 0 | 1 | 2 | 4 | Δ 2 | Δ 4 | 2 | 4 | Δ 2 | Δ 4 |
| Name | W | W | W | W | W | W | W | W | W | W |
| Aba-IV-1 | 0.0 | 0.3 | 0.3 | 0.6 | 0.3 | 0.6 | 0.1 | 0.3 | 0.1 | 0.3 |
| Aba-IV-2 | 0.0 | 0.1 | 0.2 | 3.4 | 0.2 | 3.4 | 0.0 | 0.0 | 0.0 | 0.0 |
| Aba-IV-3 | 0.0 | 0.6 | 1.3 | 0.8 | 1.3 | 0.8 | 0.0 | 0.0 | 0.0 | 0.0 |
| Aba-IV-4 | 0.0 | 0.4 | 3.7 | 7.1 | 3.7 | 7.1 | 0.1 | 0.0 | 0.1 | 0.0 |

W—indicates weeks,
T0 indicates ‘zero’ time point,
Δ—Delta indicates change in a value from zero time point to a specific time point

TABLE 4
pH of abatacept (25 mg/ml) formulations prepared
as per example 1 at 40° C. and 25° C.

| | pH | | | | |
|---|---|---|---|---|---|
| Sample | T0 | 40° C. | | 25° C. | |
| Name | 0 W | 4 W | Δ pH | 4 W | ΔpH |
| Aba-IV-1 | 7.1 | 7.3 | 0.2 | 7.3 | 0.2 |
| Aba-IV-2 | 6.8 | 8.1 | 1.3 | 6.7 | 0.1 |
| Aba-IV-3 | 7.1 | 8.5 | 1.4 | 8.6 | 1.5 |
| Aba-IV-4 | 6.8 | 7.9 | 1.1 | 8.0 | 1.2 |

W—indicates weeks,
T0 indicates ‘zero’ time point,
Δ—Delta indicates change in a value from zero time point to a specific time point

TABLE 5
Visual inspection data of abatacept (25 mg/ml) formulations prepared
as per example 1

| | Visual Inspection | | | | |
|---|---|---|---|---|---|
| Sample | T0 | 40° C. | | 25° C. | |
| Name | 0 W | 2 W | 4 W | 2 W | 4 W |
| Aba-IV-1 | Clear | Clear | Clear | Clear | Clear |
| Aba-IV-2 | Clear | Clear | Clear | Clear | Clear |
| Aba-IV-3 | Clear | Slightly | Slightly | opalescent | opalescent |
| | | opalescent | opalescent | | |
| Aba-IV-4 | Clear | opalescent | opalescent | opalescent | opalescent |

W—indicates weeks

### Example 2: High Concentration Abatacept (˜125 mg/ml) Formulations in Presence of Sugar(s) and Amino Acid(s)

Approximately 120 mg/ml of abatacept in phosphate buffer back ground obtained from tangential flow filtration (TFF) step of downstream process was buffer exchanged in the respective buffer back ground. Post which, various excipients such as sugars and amino acids were added to high concentration abatacept formulations in different combinations and concentrations. Details of the formulations are given in Table 6. FDA approved subcutaneous formulation of abatacept contains phosphate buffer, 170 mg/ml of sucrose and Poloxamer. Hence, to maintain a reference standard, to ˜120 mg/ml of in-house abatacept in phosphate buffer back ground, 170 mg/ml sucrose and 8 mg/ml of Poloxamer were added to the formulation.

All high concentration abatacept formulations were subjected for accelerated stability studies at 30° C. for two weeks. Post which, the samples were analyzed for high molecular weight (HMW) species [results are shown Table 7] using size exclusion chromatography (SEC) and also checked for change in pH [Table 8] and visual inspection [Table 9]. Light scattering of protein samples at 333 nm were also checked using nano drop and results are represented in Table 10.

TABLE 6
Compositions of various high concentration abatacept formulations
(~120 mg/ml) prepared as per example 2

| Sample | |
|---|---|
| Name | Composition |
| Aba-Ref | 120 mg/ml of Abatacept, phosphate buffer, 170 mg/ml |
| | of sucrose, 8 mg/ml poloxamer pH 6.8-7.2 |
| Aba-SC-1 | 120 mg/ml of Abatacept, phosphate buffer, 10 mg/ml |
| | histidine, 8 mg/ml poloxamer, pH 7.2 |
| Aba-SC-2 | 120 mg/ml of Abatacept, phosphate buffer, 10 mg/ml |
| | arginine, 8 mg/ml poloxamer, pH 7.2 |
| Aba-SC-3 | 120 mg/ml of Abatacept, phosphate buffer, 10 mg/ml |
| | histidine, 10 mg/ml arginine, 10 mM NaCl, 8 mg/ml |
| | poloxamer, pH 7.2 |
| Aba-SC-4 | 120 mg/ml of Abatacept, phosphate buffer, 125 mg/ml |
| | of sucrose, 5 mg/ml lysine, 10 mg/ml glycine, 10 mg/ml |
| | arginine, 10 mg/ml histidine, 8 mg/ml poloxamer, pH |
| | 7.2 |
| Aba-SC-5 | 120 mg/ml of Abatacept, phosphate buffer, 125 mg/ml |
| | of mannitol, 5 mg/ml lysine, 10 mg/ml glycine, 10 |
| | mg/ml arginine, 10 mg/ml histidine, 8 mg/ml |
| | poloxamer, pH 7.2 |
| Aba-SC-6 | 120 mg/ml of Abatacept, phosphate buffer, 125 mg/ml |
| | of sorbitol, 5 mg/ml lysine, 10 mg/ml glycine, 10 mg/ml |
| | arginine, 10 mg/ml histidine, 8 mg/ml poloxamer, pH |
| | 7.2 |
| Aba-SC-7 | 120 mg/ml of Abatacept, phosphate buffer, 125 mg/ml |
| | of sucrose, 10 mg/ml of histidine, 8 mg/ml poloxamer, |
| | pH 7.2 |
| Aba-SC-8 | 120 mg/ml of Abatacept, citrate-phosphate buffer, 125 |
| | mg/ml of mannitol, 10 mg/ml of histidine, 8 mg/ml |
| | poloxamer, pH 7.2 |

TABLE 7
SEC data of high concentration abatacept (~120
mg/ml) formulations prepared as per example 2

| | | | | High molecular | | | Low molecular | | |
|---|---|---|---|---|---|---|---|---|---|
| | Dimer content | | | weight species | | | weight species | | |
| | at 30° C. | | | at 30° C. | | | at 30° C. | | |
| Sample | 0 | 1 | 2 | 0 | 1 | 2 | 0 | 1 | 2 |
| Name | W | W | W | W | W | W | W | W | W |
| Aba-Ref | 97.8 | 92.9 | 88.7 | 2.2 | 7.1 | 11.0 | 0.0 | 0.0 | 0.3 |
| Aba-SC-1 | 97.6 | 84.8 | 75.2 | 2.4 | 15.2 | 24.8 | 0.0 | 0.0 | 0.0 |
| Aba-SC-2 | 97.6 | 89.2 | 71.3 | 2.3 | 13.0 | 28.6 | 0.0 | 0.0 | 0.2 |
| Aba-SC-3 | 97.7 | 89.7 | 79.7 | 2.3 | 10.3 | 20.1 | 0.0 | 0.0 | 0.2 |
| Aba-SC-4 | 97.6 | 79.6 | 55.5 | 2.4 | 5.8 | 17.3 | 0.0 | 14.6 | 27.2 |
| Aba-SC-5 | 97.6 | 94.5 | 86.8 | 2.4 | 4.7 | 10.7 | 0.0 | 0.8 | 2.5 |
| Aba-SC-6 | 97.6 | 87.7 | 69.7 | 2.4 | 6.5 | 12.5 | 0.0 | 0.0 | 10.1 |
| Aba-SC-7 | 97.6 | 92.4 | 86.8 | 2.4 | 7.6 | 13.2 | 0.0 | 0.0 | 0.0 |
| Aba-SC-8 | 97.7 | 94.4 | 92.6 | 2.3 | 5.6 | 7.5 | 0.0 | 0.0 | 0.0 |

W—indicates weeks

TABLE 8
pH of high concentration abatacept formulations
prepared as per example 2 at 30° C.

| Sample | pH at 30° C. | | | |
|---|---|---|---|---|
| Name | 0 W | 1 W | 2 W | Δ pH at 2 W |
| Aba-Ref | 7.0 | 6.8 | 6.8 | −0.2 |
| Aba-SC-1 | 7.3 | 7.1 | 7.2 | −0.1 |
| Aba-SC-2 | 6.9 | 6.8 | 6.8 | −0.1 |
| Aba-SC-3 | 7.3 | 7.2 | 7.2 | −0.1 |
| Aba-SC-4 | 7.1 | 6.6 | 6.6 | −0.5 |
| Aba-SC-5 | 7.1 | 7.2 | 7.8 | 0.7 |
| Aba-SC-6 | 7.1 | 6.6 | 7.4 | 0.3 |
| Aba-SC-7 | 7.3 | 7.1 | 7.1 | −0.2 |
| Aba-SC-8 | 7.5 | 7.4 | 7.4 | −0.1 |

W—indicates weeks,
Δ—Delta indicates change in a value from zero time point to a specified time point.

TABLE 9
Visual inspection data of high concentration abatacept
formulations prepared as per example 2

| Sample | Visual Inspection 30° C. | | |
|---|---|---|---|
| Name | 0 W | 1 W | 2 W |
| Aba-Ref | Clear, colorless | Clear, colorless | Clear, colorless |
| Aba-SC-1 | Clear, colorless | Clear, colorless | Clear, colorless |
| Aba-SC-2 | Clear, colorless | Clear, colorless | Clear, colorless |
| Aba-SC-3 | Clear, colorless | Clear, colorless | Clear, colorless |
| Aba-SC-4 | Clear, colorless | Clear, colorless | Clear, colorless |
| Aba-SC-5 | Clear, colorless | Clear, colorless | Clear, colorless |
| Aba-SC-6 | Clear, colorless | Clear, colorless | Clear, colorless |
| Aba-SC-7 | Clear, colorless | Clear, colorless | Clear, colorless |
| Aba-SC-8 | Clear, colorless | Clear, colorless | Clear, colorless |

W—indicates weeks

TABLE 10
Light scattering data at 333 nm (A333) of high concentration
abatacept formulations prepared as per example 3 at 30° C.

| | Light scattering data 30° C. | | |
|---|---|---|---|
| Sample Name | 0 W | 1 W | 2 W |
| Aba-Ref | 0.1 | 0.1 | 0.2 |
| Aba-SC-1 | 0.0 | 0.2 | 0.4 |
| Aba-SC-2 | 0.0 | 0.1 | 0.0 |
| Aba-SC-3 | 0.1 | 0.1 | 0.0 |
| Aba-SC-4 | 0.0 | 0.1 | 0.1 |
| Aba-SC-5 | 0.0 | 0.2 | 0.2 |
| Aba-SC-6 | 0.1 | 0.1 | 0.1 |
| Aba-SC-7 | | | |
| Aba-SC-8 | 0.1 | 0.1 | 0.0 |

### Example 3: Addition of Excipients During Tangential Flow Filtration (TFF) Step on Stability of High Concentration CTLA4-Ig Fusion Proteins

In example 2, purified CTLA4-Ig obtained from the downstream chromatographic step was further buffer exchanged into phosphate buffer and concentrated by tangential flow filtration (TFF). Post which, excipients were added to the formulations. However, differing from this conventional strategy, various sugars such as sucrose and mannitol and amino acid such as histidine and glycine were incorporated during the TFF itself (i.e., before the formulation step or before formulating the protein as a drug product). 8-15 mg/ml concentration of abatacept fusion protein in acetate buffer obtained from chromatographic step was subjected for ultrafiltration to concentrate up to 60 mg/ml. Post which, the samples were subjected for diafiltration wherein diafiltration medium contained phosphate buffer (formulation buffer) without and with excipients such as sugars and amino acid(s), and in another separate experiment, the diafiltration medium without sugar and amino acid(s) in the phosphate buffer was experimented. Post diafiltration, the samples were subjected for second ultrafiltration to concentrate up to 180 mg/ml to 200 mg/ml. These high concentration samples were found to be stable without any visible particles/aggregates. The highly concentrated samples were further diluted to 125 mg/ml and some of the excipients such as sugars, surfactant and optionally amino acid such as glycine was added to prepare a final formulation. 8 mg/ml of poloxamer was added to all final formulation. Details of the formulations are given in Table 11. Post which, all the samples were subjected for accelerated stability studies at 30° C. for 2 weeks. The samples were analyzed for high molecular weight (HMW) species and active dimer form [results are shown Table 12] using size exclusion chromatography (SEC) and also checked for change in pH [Table 13] and visual inspection [Table 14].

TABLE 11
Compositions of various high concentration abatacept formulations
prepared as per example 3

| Sample | Formulation buffer | Formulation composition after |
|---|---|---|
| Name | composition during TFF | TFF |
| Aba-SC-9 | 20 mM phosphate buffer | 125 mg/ml of Abatacept, 20 |
| | | mM phosphate buffer, 8 mg/ml |
| | | poloxamer, pH 7.2 |
| Aba-SC-10 | 20 mM phosphate buffer | 125 mg/ml of Abatacept, 20 |
| | and 75 mg/ml Sucrose | mM phosphate buffer, 170 |
| | | mg/ml sucrose, 8 mg/ml |
| | | poloxamer, pH 7.2 |
| Aba-SC-11 | 20 mM phosphate buffer | 125 mg/ml of Abatacept, 20 |
| | and 75 mg/ml Sucrose | mM phosphate buffer, 100 |
| | | mg/ml sucrose, 10 mg/ml |
| | | lysine, 0.58 mg/ml NaCl, 8 |
| | | mg/ml poloxamer, pH 7.2 |
| Aba-SC-12 | 20 mM phosphate buffer, | 125 mg/ml of Abatacept, 20 |
| | 75 mg/ml mannitol | mM phosphate buffer, 75 mg/ml |
| | | mannitol, 6.6 mg/ml ammonium |
| | | sulphate, 8 mg/ml poloxamer, |
| | | pH 7.2 |
| Aba-SC-13 | 20 mM phosphate buffer, | 125 mg/ml of Abatacept, 20 |
| | 75 mg/ml mannitol | mM phosphate buffer, 100 |
| | | mg/ml mannitol, 10 mg/ml |
| | | Glycine, 8 mg/ml poloxamer, |
| | | pH 7.2 |
| Aba-SC-14 | 20 mM phosphate buffer; | 125 mg/ml of Abatacept, 20 |
| | 75 mg/ml mannitol, 10 | mM phosphate buffer, 75 mg/ml |
| | mg/ml histidine | mannitol, 10 mg/ml histidine, 8 |
| | | mg/ml poloxamer, pH 7.2 |
| Aba-SC-15 | 20 mM phosphate buffer; | 125 mg/ml of Abatacept, 20 |
| | 75 mg/ml mannitol, 15 | mM phosphate buffer, 75 mg/ml |
| | mg/ml histidine | mannitol, 15 mg/ml histidine, 8 |
| | | mg/ml Poloxamer, pH 7.2 |
| Aba-SC-16 | 20 mM phosphate buffer; | 125 mg/ml of Abatacept, 20 |
| | 75 mg/ml mannitol, 10 | mM phosphate buffer, 75 mg/ml |
| | mg/ml histidine | mannitol, 10 mg/ml histidine, 5 |
| | | mg/ml glycine, 8 mg/ml |
| | | poloxamer, pH 7.2 |
| Aba-SC-17 | 20 mM phosphate buffer; | 125 mg/ml of Abatacept, 20 |
| | 75 mg/ml mannitol, 15 | mM phosphate buffer, 75 mg/ml |
| | mg/ml histidine | mannitol, 15 mg/ml histidine, 5 |
| | | mg/ml glycine, 8 mg/ml |
| | | poloxamer, pH 7.2 |
| Aba-SC-18 | 20 mM phosphate buffer; | 125 mg/ml of Abatacept, 20 |
| | 85 mg/ml mannitol, 10 | mM phosphate buffer, 85 mg/ml |
| | mg/ml histidine | mannitol, 10 mg/ml histidine, 8 |
| | | mg/ml poloxamer, pH 7.2 |
| Aba-SC-19 | 20 mM phosphate buffer; | 125 mg/ml of Abatacept, 20 |
| | 85 mg/ml mannitol, 10 | mM phosphate buffer, 85 mg/ml |
| | mg/ml histidine | mannitol, 10 mg/ml histidine, 5 |
| | | mg/ml glycine, 8 mg/ml |
| | | poloxamer, pH 7.2 |
| Aba-SC-20 | 20 mM phosphate buffer; | 125 mg/ml of Abatacept, 20 |
| | 85 mg/ml mannitol, 15 | mM phosphate buffer, 85 mg/ml |
| | mg/ml histidine | mannitol, 15 mg/ml histidine, 8 |
| | | mg/ml poloxamer, pH 7.2 |
| Aba-SC-21 | 20 mM phosphate buffer, | 125 mg/ml of Abatacept, 20 |
| | 75 mg/ml mannitol, 10 | mM phosphate buffer, 75 mg/ml |
| | mg/ml histidine | mannitol, 10 mg/ml histidine, |
| | | 10 mg/ml proline, 8 mg/ml |
| | | poloxamer, pH 7.2 |

TABLE 12
SEC data of high concentration abatacept
formulations prepared as per example 3

| | | | High molecular | | Low molecular | |
|---|---|---|---|---|---|---|
| | Dimer content | | weight species | | weight species | |
| | at 30° C. | | at 30° C. | | at 30° C. | |
| Sample | 0 | 2 | 0 | 2 | 0 | 2 |
| Name | W | W | W | W | W | W |
| Aba-SC-9 | 98.6 | 59.7 | 1.4 | 37.6 | 0 | 2.7 |
| Aba-SC-10 | 98.6 | 87.6 | 1.4 | 12.4 | 0 | 0 |
| Aba-SC-11 | 98.7 | 75.8 | 1.3 | 24.2 | 0 | 0 |
| Aba-SC-12 | 98.2 | 87.3 | 1.8 | 12.6 | 0 | 0.1 |
| Aba-SC-13 | 98.9 | 88.2 | 1.2 | 11.9 | 0 | 0 |
| Aba-SC-14 | 98.8 | 91.2 | 1.2 | 8.8 | 0 | 0 |
| Aba-SC-15 | 98.9 | 92.0 | 1.1 | 8.1 | 0 | 0 |
| Aba-SC-16 | 98.8 | 91.7 | 1.2 | 8.3 | 0 | 0 |
| Aba-SC-17 | 98.9 | 92.4 | 1.1 | 7.6 | 0 | 0 |
| Aba-SC-18 | 98.9 | 91.7 | 1.1 | 8.3 | 0 | 0 |
| Aba-SC-19 | 98.9 | 92.0 | 1.1 | 8.0 | 0 | 0 |
| Aba-SC-20 | 98.8 | 92.3 | 1.2 | 7.7 | 0 | 0 |
| Aba-SC-21 | 99.0 | 87.5 | 1.0 | 12.5 | 0 | 0 |

TABLE 13
pH data of high concentration abatacept
formulations prepared as per example 3

| Sample | pH at 30° C. | | |
|---|---|---|---|
| Name | 0 W | 2 W | Δ pH at 2 W |
| Aba-SC-9 | 6.9 | 6.1 | −0.8 |
| Aba-SC-10 | 7.0 | 6.9 | −0.1 |
| Aba-SC-11 | 6.8 | 6.8 | 0 |
| Aba-SC-12 | 7.0 | 7.1 | 0.1 |
| Aba-SC-13 | NT | NT | NA |
| Aba-SC-14 | 7.0 | 6.9 | −0.1 |
| Aba-SC-15 | 7.0 | 7.1 | 0.1 |
| Aba-SC-16 | 7.1 | 7.2 | 0.1 |
| Aba-SC-17 | 7.0 | 7.1 | 0.1 |
| Aba-SC-18 | 7.1 | 7.2 | 0.1 |
| Aba-SC-19 | 7.0 | 7.1 | 0.1 |
| Aba-SC-20 | 7.0 | 7.1 | 0.1 |
| Aba-SC-21 | 7.1 | 7.2 | 0.1 |

W—indicates-weeks,
NT—Not tested due to sample constrain;
NA—not applicable

TABLE 14
Visual inspection data of high concentration abatacept formulations
prepared as per example 3

| Sample | Visual Inspection at 30° C. | |
|---|---|---|
| Name | 0 W | 2 W |
| Aba-SC-9 | Clear | Turbid |
| Aba-SC-10 | Clear, colorless, no visible | Clear, colorless, no visible |
| | particles | particles |
| Aba-SC-11 | Clear, colorless, no visible | Clear, colorless, no visible |
| | particles | particles |
| Aba-SC-12 | Clear, colorless, no visible | Clear, colorless, no visible |
| | particles | particles |
| Aba-SC-13 | Clear, colorless, no visible | Clear, colorless, no visible |
| | particles | particles |
| Aba-SC-14 | Clear, colorless, no visible | Clear, colorless, no visible |
| | particles | particles |
| Aba-SC-15 | Clear, colorless, no visible | Clear, colorless, no visible |
| | particles | particles |
| Aba-SC-16 | Clear, colorless, no visible | Clear, colorless, no visible |
| | particles | particles |
| Aba-SC-17 | Clear, colorless, no visible | Clear, colorless, no visible |
| | particles | particles |
| Aba-SC-18 | Clear, colorless, no visible | Clear, colorless, no visible |
| | particles | particles |
| Aba-SC-19 | Clear, colorless, no visible | Clear, colorless, no visible |
| | particles | particles |
| Aba-SC-20 | Clear, colorless, no visible | Clear, colorless, no visible |
| | particles | particles |
| Aba-SC-21 | Clear, colorless, no visible | Clear, colorless, no visible |
| | particles | particles |

Viscosity of some of the abatacept formulations (Aba-SC-14 and Aba-SC-18) prepared as per example 3 were measured using m-VROC® viscometer and viscosity of the formulations (Aba-SC-14 and Aba-SC-18) were found to be 9.0 mPa/S.

### Example 4: Colloidal Stability of High Concentration Abatacept Protein Formulations

Abatacept, formulations containing sugar and amino acid were subjected for dynamic light scattering (DLS) to measure the diffusion co efficient, hydrodynamic diameter which is indicative of solubility of abatacept. DLS measurements in turn gives information about colloidal stability of a protein molecule. Results of the same are represented in Table-15.

TABLE 15
Dynamic light scattering (DLS) data of high concentration abatacept
formulations prepared as per example 3

| | | Hydrodynamic | | Diffusion | |
|---|---|---|---|---|---|
| | | radius (Rh) | | co-efficient | |
| | | (nm) | | (Cm²/s) | |
| Sample | | | 4 W at | | 4 W at |
| Name | Composition | 0 W | 30° C. | 0 W | 30° C. |
| Aba-SC-14 | 125 mg/ml of | 5.7 | 5.9 | 4.34E−07 | 4.20E−07 |
| | Abatacept, 20 mM | | | | |
| | phosphate buffer, 75 | | | | |
| | mg/ml mannitol, 10 | | | | |
| | mg/ml histidine, 8 | | | | |
| | mg/ml poloxamer, | | | | |
| | pH 7.2 | | | | |
| Aba-SC-18 | 125 mg/ml of | 5.6 | 5.4 | 4.37E−07 | 4.60E−07 |
| | Abatacept, 20 mM | | | | |
| | phosphate buffer, 85 | | | | |
| | mg/ml mannitol, 10 | | | | |
| | mg/ml histidine, 8 | | | | |
| | mg/ml poloxamer, | | | | |
| | pH 7.2 | | | | |
| Aba-SC-22 | 125 mg/ml of | 10.0 | 12.7 | 2.49E−07 | 2.00E−07 |
| | abatacept, 20 mM | | | | |
| | phosphate buffer, | | | | |
| | 100 mg/ml sucrose, | | | | |
| | 10 mg/ml histidine, | | | | |
| | 8 mg/ml Poloxamer | | | | |

1. A stable aqueous pharmaceutical formulation of a CTLA4-Ig fusion protein comprising, CTLA4-Iq fusion protein, a buffer, polyol, amino acid and surfactant, and wherein the formulation is devoid of sucrose.

2. The formulation according to claim 1, wherein the ratio of CTLA4-Ig fusion protein to polyol is 1:0.7 or lower and the ratio of CTLA4-Ig fusion protein to amino acid is 1:0.1 or lower.

3. The formulation according to claim 1, wherein the concentration of CTLA4-Ig fusion protein formulation is from about 20 mg/ml to about 200 mg/ml.

4. The formulation according to claim 1, wherein the pH of the formulation is from 6.0 to 8.0.

5. The formulation according to claim 1, wherein the formulation has a viscosity of less than 15 cp.

6. The formulation according to claim 1, wherein
the fusion protein in formulation is stable at 30° C. for at least two weeks and contains less than about 10% of fusion protein molecule in aggregate form.

7. A stable formulation of CTLA4-Ig fusion protein comprising phosphate buffer, mannitol, histidine and surfactant, and wherein the formulation is devoid of sucrose.

8. The formulation according to claim 7, wherein the concentration of mannitol is about 80 mg/ml and the concentration of histidine is between 10 mg/ml-15 mg/ml.

9. A method of obtaining a stable formulation of CTLA4-Ig fusion protein comprising addition of buffer, polyol, histidine and surfactant to the CTLA4-Ig fusion protein, and wherein histidine and polyol are also added to the pre-formulation process steps of ultrafiltration and diafiltration steps containing the fusion protein.

10. A method of increasing the stability of CTLA4-Ig fusion protein comprising steps of, expression and purification of CTLA4-Ig fusion protein; concentration and/or buffer exchange of the protein by ultrafiltration and diafiltration (UF-DF), wherein the buffer used in the UF and/or DF step(s) include histidine and polyol; followed by formulation of the protein in a buffer comprising histidine, polyol and surfactant; wherein the stability of the protein is increased compared to the formulation of the protein that was processed by UF-DF steps without the inclusion of histidine and polyol in its buffer.
